(19) United States
(12) Patent Application Publication
(10) Pub. No.: US 2005/0175597 A1
(43) Pub. Date: Aug. 11, 2005

(54) Compositions containing labile bioactive materials and mammalian colostrum, methods of preparation and treatment

(76) Inventors: Grant Thomas Rawlin, Kilmore East (AU); Gottfried Lichti, Essendon (AU); Roy Michael Robins-Browne, Templestowe (AU)

Correspondence Address: Michael L Goldman;Nixon Peabody, Clinton Square, PO Box 31051, Rochester, NY, 14603, US

(21) Appl. No.: 10/508,172

(22) PCT Filed: Mar. 21, 2003
(86) PCT No.: PCT/AU03/00348

(30) Foreign Application Priority Data
Mar. 21, 2002 (AU) PS 1278
May 24, 2002 (AU) PS 2551

(51) Int. Cl. A61K 39/02; A61K 35/20

(52) U.S. Cl. 424/93.45; 424/234.1; 424/535

(57) ABSTRACT

The invention relates t a method of improving the viability of a labile bioactive substance on administration to a hostile environment, the method comprising forming a mixture of the bioactive substance and mammalian colostrum and also a composition for administration of a labile bioactive substance the composition comprising a mixture of the bioactive substance and mammalian colostrum.

### BACKGROUND

Bioactive substances, which may have a powerful physiological effect, are frequently very sensitive to hostile environments such as the mammalian gastric environment, a high temperature environment, or a high-humidity environment. This frequently limits their use and means they cannot be conveniently administered orally, or they can not be conveniently stored, or they can not be conveniently applied on a warm product line.

The bioactive substances may be selected from the group including growth promoters, antineoplastic agents, oral vaccines, inhalants, living microorganisms (for example protobiotics such as Lactobacillus spp), peptides, polypeptides, nucleotides, polynucleotides, nucleosides, proteins, glycoproteins, sugars and complex carbohydrates, anti-infectants, antimicrobials, disinfectants, antiseptics, antidepressants, psychoactive agents, genetically modified organisms and infectious agents used as vectors for other bioactive substances eg bacterial vectors (including E. coli, Salmonella, Vibrio, Lactobacilli, Bacillus, Mycobacteria, Shigella), viral vectors (including Adenovirus, Poxvirus, Bacculovirus, Herpesvirus, Enterovirus, Paramyxovirus and Orthomyxovirus), plant vectors (including tobacco, potato and banana), yeast vectors, immunoglobulins or affinity purified immunoglobulins including antibodies directed against diseases and disease causing agents (for example Helicobacter pylori, E. coli, Bacillus spp, pathogenic Yersinia spp., and allergens) and fragments, derivatives and complexes containing any of the above.

Examples of hostile regions which compromise the function of bioactive substances include highly acid or highly alkaline regions, regions which contain proteolytic enzymes, regions in which desiccation occurs, regions of high humidity, regions of high temperature, regions in which elevated pressure leads to denaturation eg a tableting machine and regions containing DNA-ases.

A particularly hostile region for compromising the function of bioactive substances is a mammalian gastric environment, which contains areas of high acid conditions, elevated temperature, moisture, high concentrations of proteolytic enzymes and carbohydrate digesting enzymes.

A particular application of this invention is to the preservation of bioactive agents whose biological function is compromised in a mammalian gastric environment.

A number of methods to preserve bioactive function in a mammalian gastric environment are known to the art. These include enteric coatings, buffering, formalin stabilisation, addition of antisecretory agents, giving the substance in conjunction with a meal and altering the immunochemistry of cows to cause the secretion of bioactive materials into colostrum.

Freichel O L and Lippold B C (International Journal of Pharmacology, Mar. 23, 2001; 216(1-2): 165-9) suggest the use of an enteric coating comprising methylhydroxy ethylcellulose and hydroxypropyl methlycellulose acetate succinate to provide a coating that protects its contents in mammalian stomachs.

Tacket C et al (The New England Journal of Medicine, May 12, 1986, 1240-1243) used a buffering solution of bicarbonate of soda to decrease proteolysis in the human stomach.

Paliwal R and London E (Biochemistry Feb. 20, 1996; 35 (7): 2374-9) describe the use of formalin treatment to stabilise the conformation of diphtheria toxoid.

Asad M et al (Life Science Nov. 21, 2001; 70(1):17-24) used oxytocin and rantinide respectively to decrease acid secretion in the stomach of mammals to increase gastric ulcer healing rates.

McClead and Gregory, Infections and Immunity 44: 474478 have shown that specific proteins secreted by cows into colostral milk are more stable in the gastric environment than would be expected when proteins of the same class are produced by alternative processes This principle of preserving bioactive function has been utilised by Abbot U.S. Pat. No. 5,260,037, who immunised cows prior to harvesting of colostral milk. The immunisation leads to the secretion of specific antibodies (eg directed against Helicobacter pylori) into colostral milk (this is called hyperimmune colostrum).

Hyperimmune bovine colostrum has also been described in International Application PCT/AU94/00562 to be stable in terms of biological function in high pressure environments for example in a tableting machine. The use of hyperimmune bovine colostrum has also been taught by:
Mitra et al, Acta Paediatrica, 84: 996-1001, 1995. Hyper immune cow colostrum reduces diarrhoea due to rotavirus: a double blind, controlled clinical study.
Nord et al, AIDS 4, 581-584, 1990. Treatment with bovine hyperimmune colostrum of cryptosporidial diarrhea in AIDS patients.
Tacket et al, New England Journal of Medicine. Ma7 12, 1988. Protection by milk immunoglobulin concentrate against oral challenge with enterotoxigenic E. coli.
Tacket et al, American Journal of Tropical Medical Hygiene. 47 (3), 276-283. 1992. Efficacy of Bovine milk immunoglobulin concentrate in preventing illness after Shigella flexeri challenge.

In the above prior art the hyperimmune colostrum was the source of bioactive material and subsequent operations (if any) involved the removal of undesirable components eg water, fat, cellular material, bacteria and lactose. There is no teaching that the addition of colostrum or colostrum extract or colostrum components leads to enhanced protection of the function of the bioactive substance in a hostile environment.

Problems associated with the above methods for preserving bioactive function in a mammalian gastric environment include the following:

The strategy of using enteric coatings is inappropriate for delivering agents which act in the stomach. This is a particular limitation with respect to the control of Helicobacter pylori infection (which is a significant cause of gastritis) by oral vaccines and/or the use of antibodies directed against H. pylori in a passive immunity approach.

The strategy of using buffer solutions to decrease proteolysis in a mammalian stomach is inappropriate when the loss of function in the bioactive material is occasioned by the action of enzymes eg peptidases, amylases. Such enzymes are frequently associated with loss of function in protein therapeutics and in therapeutics based on living organisms. Furthermore, the use of buffers such as bicarbonate on a regular basis is undesirable.

The use of formalin treatment (or other cross-linking strategies) to preserve bioactive function in a hostile environment is frequently ineffective when the cross-linking reaction destroys the structure and function of the substance. For this reason this technique is rarely used except present a surface antigen to stimulate an immune reaction (eg Diphtheria toxin as discussed by Petre et al Developmental Biological Standards 1996; 87: 125-34).

The method of encouraging the secretion of bioactive material by cows into colostral milk has a number of limitations.

Some bioactive materials are difficult or impossible for cows to secrete into colostrum.

The amount of bioactive material is variable and this is frequently unacceptable in quality control terms.

The maximum amount of specific bioactive material is also low and typically less than 5% of immunoglobulins in colostrum. The acceptable maximum volume in a regimen of tablets is 0.5 cubic centimetre per tablet (2 to 3 tablets per day is acceptable). Low concentrations of specific antibodies severely limit therapeutic options.

Furthermore the immunised cows that produce colostrum also produce milk for human consumption. This presents difficulties in terms of regulatory and food safety issues.

Furthermore, colostrum is harvested only once a year from a cow at calving and this causes problems in logistics and costs of production, particularly if the bioactive ingredient is secreted by the cow into the colostrum.

### SUMMARY

We have made the surprising discovery that it is possible to preserve the function of a labile bioactive substance by combining the labile bioactive substance with mammalian colostrum or components thereof in an in vitro (outside the body) mixing operation. The mammalian colostrum may be a processed form of mammalian colostrum.

The invention accordingly provides a composition for administration of a labile bioactive substance comprising a mixture of the labile bioactive substance and a mammalian colostrum.

A labile bioactive substance is a bioactive substance which is functionally impaired in its environment of use particularly in the case of hostile environment such as a mammalian stomach or rumen, a high temperature environment or high humidity environment.

In a further aspect the invention provides a method of administering a labile bioactive substance (for example a bioactive substance which is functionally impaired in the mammalian stomach or other hostile environment) comprising forming a mixture of the bioactive substance and mammalian colostrum and applying the composition to an environment under which the bioactive substance is otherwise labile such as a gastric environment or other hostile environment.

In one preferment the composition is administered orally.

The invention further provides a method for using a bioactive substance which is labile, that is functionally impaired, in the mammalian stomach or other hostile environment, in the manufacture of an ingestible medicament for treating or preventing disease, comprising mixing the therapeutic substance with mammalian colostrum or components thereof.

Although greater than expected stability of substances secreted by cows into colostrum has been noted by many workers, there has been no suggestion that colostrum or processed colostrum can be added to bioactive materials to preserve their function in the stomach or rumen or other hostile environment. Preferably the bioactive substance is a substance capable of causing a measureable change in a physiological or pharmaceutical parameter of an organism.

In one particularly preferred embodiment the bioactive substance is an antibiotic. Accordingly the invention provides an antibiotic composition comprising an antibiotic and mammalian colostrum and optionally excipients. In a further aspect of the invention we provide a probiotic composition comprising a probiotic bacterium such as Lactobacillus spp. and mammalian colostrum.

### DETAILED DESCRIPTION

The invention is preferably used with a bioactive substance which shows at least 20% diminution in function after the substance has been incubated for 60 minutes at 37° C. in a liquor comprising a 0.32% solution of porcine pepsin in 0.03 M NaCl and adjusted to pH 1.2 with HCl.

Preferably the mammalian colostrum is bovine colostrum retained from the first 4 days post parturition, more preferably bovine colostrum retained from the first 2 days post parturition, even more preferably bovine colostrum retained from the first day post parturition, and most preferably bovine colostrum retained from the first milking post parturition.

The term colostrum where used herein includes colostral milk; processed colostral milk such as colostral milk processed to partly or completely remove one or more of fat, cellular debris, lactose and casein; and colostral milk or processed colostral milk which has been dried by for example, freeze drying, spray drying or other methods of drying known in the art. Colostral milk is generally taken from a mammal such as a cow within five days after parturition.

Preferably the mammalian colostrum has been processed using a defatting operation, more preferably using a defatting operation and an operation to remove cellular debris, more preferably a defatting operation, an operation to remove cellular debris and an operation to remove salts, sugars, other low molecular weight entities and some water.

The colostrum and or bioactive material may be in dried form. The components may be intimately mixed before, during or after the drying process.

Preferably the bioactive substances and mammalian colostrum are mixed, and the mixture is an aqueous mixture which may be dried preferably by lyophilization.

In one preferment a mixture of the bioactive substance and colostrum extract is lyophilised and at least half of the lyophilised material by weight comprises added colostrum or processed colostrum. In another preferment at least three quarters of the lyophilised material by weight comprises colostrum or processed colostrum.

Preferably the bovine colostrum collected from the cow comprises at least 4% total protein (weight %), more preferably 5%, more preferably at least 8%, more preferably at least 10%.

Preferably the ratio of IgG to total protein of the colostrum collected from the cow is at least 10%, more preferably 20%.

Preferably the bioactive substance is selected from the group consisting of growth promoters, antineoplastic agents, oral vaccines, inhalants, living microorganisms (for example protobiotics such as Lactobacillus spp), peptides, polypeptides, nucleotides, polynucleotides, nucleosides, proteins, glycoproteins, sugars and complex carbohydrates, anti-infectants, antimicrobials, disinfectants, antiseptics, antidepressants, psychoactive agents, genetically modified organisms and infectious agents used as vectors for other bioactive substances eg bacterial vectors (including E. coli, Salmonella, Vibrio, Lactobacilli, Bacillus, Mycobacteria, Shigella), viral vectors (including Adenovirus, Poxvirus, Bacculovirus, Herpesvirus, Enterovirus, Paramyxovirus and Orthomyxovirus), plant vectors (including tobacco, potato and banana), yeast vectors, immunoglobulins, affinity purified immunoglobulins including antibodies directed against diseases and disease causing agents (for example Helicobacter pylori, E. coli, Bacillus spp, pathogenic Yersinia spp., and allergens) and fragments, derivatives and complexes containing any of the above.

It is particularly preferred that the bioactive material comprises monoclonal or polyclonal immunoglobulins or chimeric monoclonal antibodies or humanised monoclonal antibodies or dendrimer presented immunoactive fragments or immunoactive fragments such as F(ab) and F(ab)2 fragments or recombinant immunoactive fragments, or affinity purified immunoglobulins or immunoactive fragments thereof. These immunoglobulins or fragments thereof may bind to pathogenic organisms including Helicobacter pylori, Enterotoxigenic E. coli, Yersinia pestis, enterovirus 71.

The proportions of colostrum and bioactive substance in the compositions of the invention will depend on the nature of the active substance and the degree to which it is sensitive to conditions in the stomach. Typically the weight ratio of colostrum to active substance is greater than 0.5:1, more preferably greater than 2:1, even more preferably greater than 5:1. The upper limit of the added colostrum component is constrained by the practical limit of the amount of therapeutic substance which can be conveniently administered.

The composition of the invention may be administered in a range of forms such as capsule, powder tablets, aerosol spray, syrup, liquid or other form known in the art. The composition may further comprise carriers or excipients suitable for gastrointestinal administration. Examples of carriers and excipients include: silica, talc, titanium dioxide, alumina, starch, kaolin, powdered cellulose, microcrystalline cellulose, Amylopectin N, sucrose, lactose, dextrose, polyvinylpyrrolidone, hydroxypropyl cellulose, methyl cellulose, hydroxyethyl cellulose, carboxymethyl cellulose, citric acid, sodium bicarbonate, magnesium stearate, shellac, cellulose acetate, cetyl alcohol, triethyl citrate, polyethylene glycol.

In another particular preferment the bioactive material comprises immunogenic proteins, glycoproteins or other components of a vaccine or organisms which express components of a vaccine. The vaccine may be directed at a pathogenic organism for example Helicobacter pylori, Enterotoxigenic E. coli, Yersinia pestis.

In a particularly preferred embodiment the bioactive substance is selected from the group consisting of:
(a) antibodies and fragments thereof against the disease organisms H. pylori, enterotoxic E. coli (ETEC), picoma viruses especially Enteroviruses rotavirus anthrax and Yersinia pestis;
(b) antibodies and fragments thereof against toxins produced by H. pylori, ETEC, anthrax, Yersinia pestis and antibodies against ricin; and
(c) oral vaccines and inhalation vaccines against H. pylori, ETEC, picoma viruses especially Enteroviruses, rotavirus, anthrax, Yersinia pestis.

In a particularly preferred embodiment the invention provides a method of treatment or prophylaxis of gastrointestinal disease in a patient comprising administering to the patient a composition comprising (a) a vaccine directed against a pathogenic microorganism selected from H. pylori and coliforms and (b) mammalian colostrum. The colostrum will generally be present in an amount sufficient to enhance the activity of the vaccine in the gastric environment.

In another particularly preferred embodiment the invention provides a method of treatment or prophylaxis of gastric ulcers or H. pylori infection in a patient comprising administering to the patient a composition comprising a binding protein directed to H. pylori and further comprising mammalian colostrum.

In one preferment the immunoglobulins or fragments thereof are derived from eggs, preferably from hens immunised with killed vaccines against pathogens.

The invention is particularly useful for delivering bioactive agents which act in the stomach. For example agents against Helicobacter pylori infection. However even bioactive substances used to control respiratory pathogens in the form of an inhaled spray may lose activity when placed in simulated gastric fluid or other hostile environment. The colostrum or processed colostrum may protect against this loss of function, and this protective effect is expected to be beneficial when the bioactive substance is delivered as an inhaled spray.

The invention is useful for protecting bioactive agents from proteolysis occasioned by enzyme action as well as proteolysis occasioned by low pH conditions.

The field of probiotics has developed rapidly over the last 10 years. Probiotics comprise beneficial bacteria that may out complete harmful bacteria in the gastrointestinal tract. The indigestion of friendly bacteria discourage the presence of Lactobacillus spp. and Bifidobacter spp are important bacteria used in commercial probiotics sold in Australia. Lactic acid production in these bacteria discourage the harmful bacteria which do not thrive in acid environments. Lactobacillus may also produce specific antibiotics. For example Lactobacillus acidophilus produces acidophiline and L. bugaricus produced bulgarican. Bifidobacterium can also be used as a probiotic organism. It has been reported that probiotics aid in digestion, assist in cleaning the gut and promoting regular bowel movements, contribute to the destruction of mould, viruses and parasites and balance intestinal pH.

Probiotics are also useful in treatment or prophylaxis of acid gut syndrome resulting from accumulation of acid and production of endotoxin in the gastrointestinal tract.

Administration of probiotics orally can result in significant loss of activity as a result of the probiotic passing through the severe conditions in the stomach. We have found that a greater level of activity of the probiotic is preserved when administered orally in accordance with the invention.

The probiotic may contain excipients and adjuvants and may be in tablet, powder, capsule or liquid form. The invention further provides a method of treatment or propylaxis of gastrointestinal dysfunction comprising administering a composition comprising a probiotic bacteria such as Lactobacillus spp. and mammalian colostrum.

The dose of bacterium may depend on the condition of the patient and nature of the dysfunction but is typically at least 10⁵ CFU Lactobacillus per day.

The invention is useful for preserving the function of non-cross-linked bioactive materials.

The invention enables the formulation of therapeutic agents which are significantly superior to hyperimmune colostrum in the following respects:
The amount of bioactive material can be controlled to within tight limits
The amount of bioactive material, which is prepared separately to the colostrum, is no longer subject to variable production of immunoactive material by the cows. This is highly desirable from a quality control point of view.
The amount of bioactive material that can be presented in a given volume can be increased over that possible with hyperimmune colostrum by a factor greater than 4. For example hyperimmune immunoglobulin from avian eggs particularly an egg yolk from an immunised hen can be affinity purified and F(ab)₂ fragments made. This provides significantly expanded therapeutical options.
The invention allows the production of therapeutics without compromising the integrity of dairy production for human consumptions. For example the bioactive active substance can be manufactured in the laboratory and the colostrum harvested from normal cows.
Bovine Colostrum extract is a substance listed by the Therapeutic Goods Authority for use as a complementary medicine in Australia so that utilisation of colostrum in oral therapeutics is facilitated.

The invention will now be described with reference to the following examples. It is to be understood that the examples are provided by way of illustration of the invention and that they are in no way limiting to the scope of the invention.

Many of the examples are discussed with reference to results shown in the attached drawings. In the drawings:

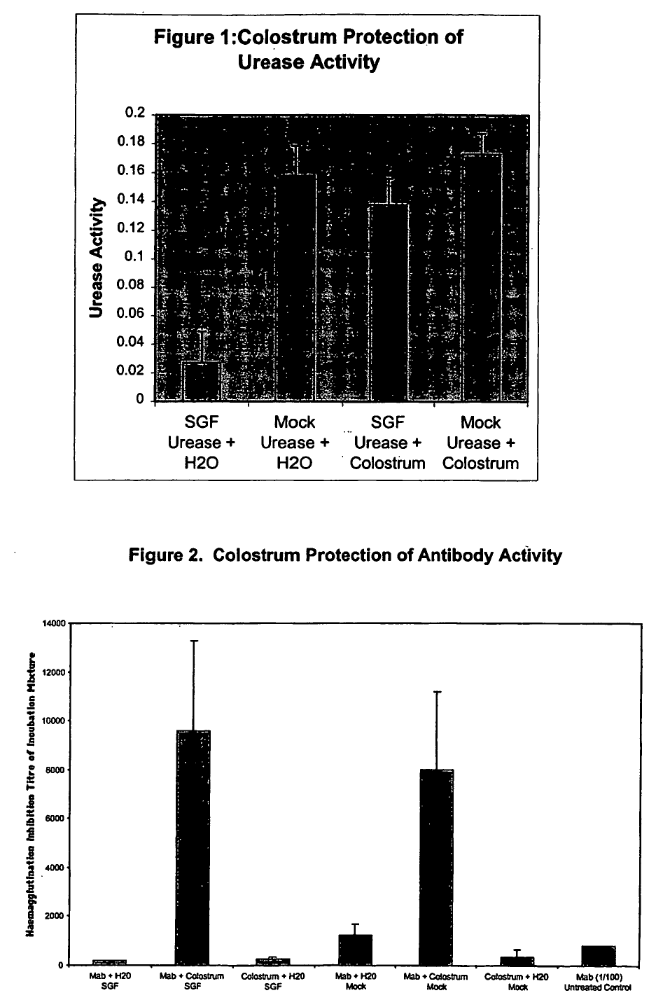

FIG. 1 is a chart showing the colostrum protection of urease activity reported in Example 1;

FIG. 2 is a chart showing the colostrum protection of antibody activity reported in Example 3;

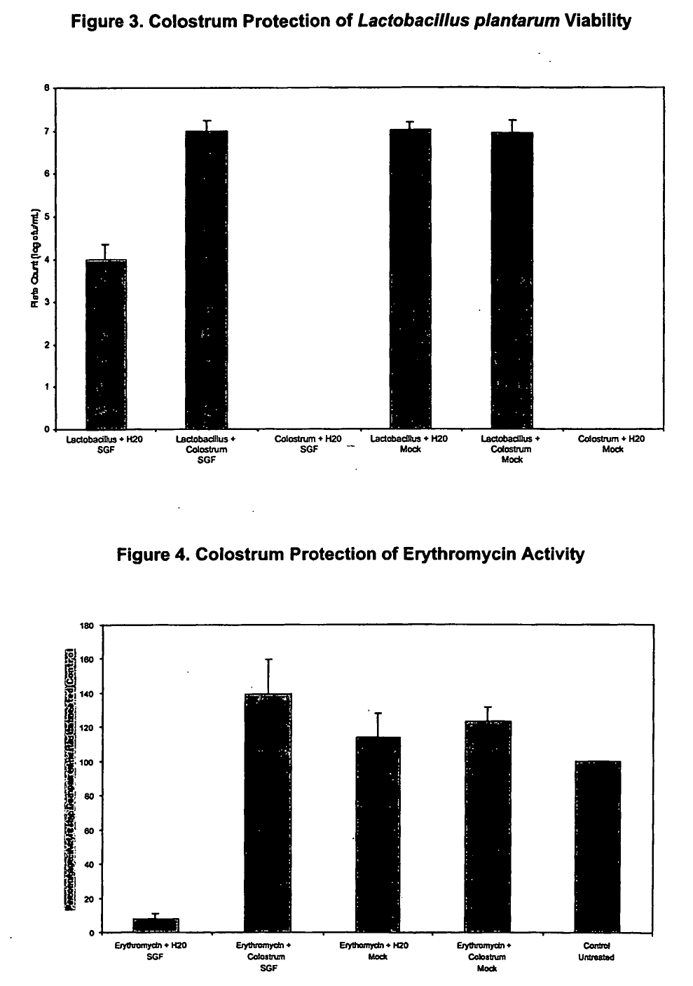

FIG. 3 is a chart showing colostrum protection of Lactobacillus plantarum viability reported in Example 4;

FIG. 4 is a chart showing colostrum protection of erythromycin activity reported in Example 6;

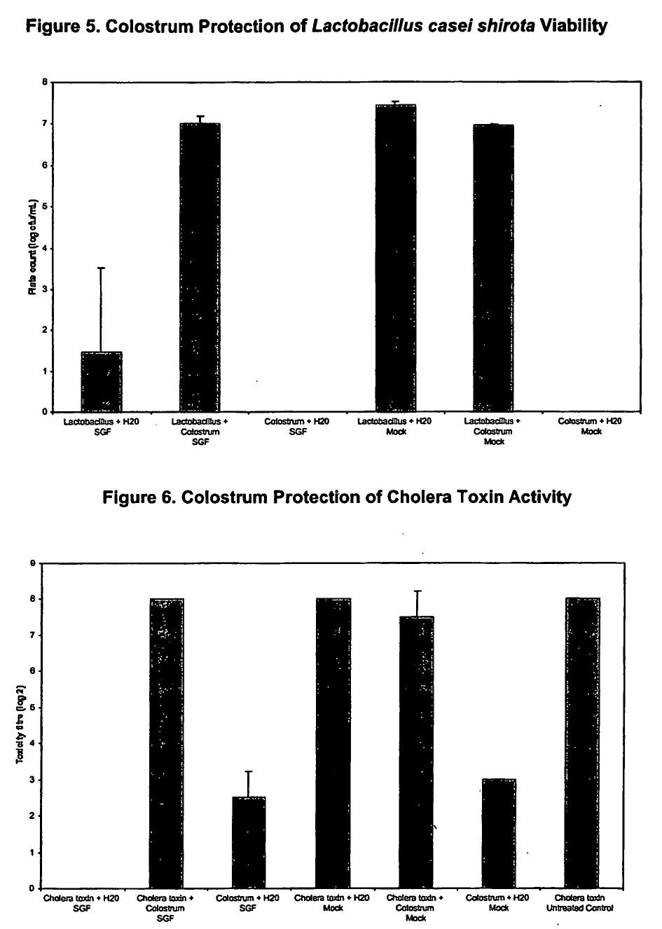

FIG. 5 is a chart showing colostrum protection of Lactobacillus casei shirota viability reported in Example 7;

FIG. 6 is a chart showing colostrum protection of chlolera toxin activity reported in Example 8;

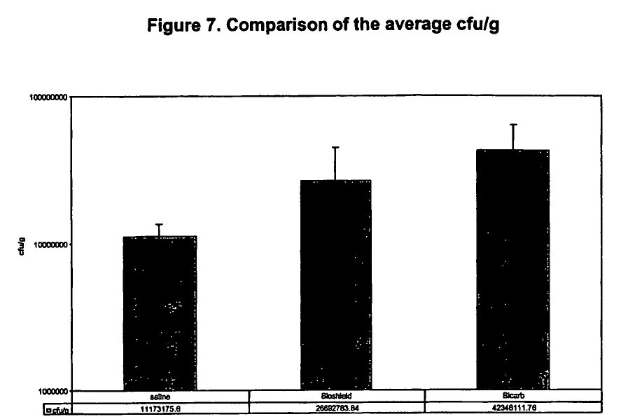

FIG. 7 is a chart showing the colostrum protection of L. plantarum reported in Example 9.

### EXAMPLES

### Example 1

Protection by colostrum of the activity of an enzyme within the stomach environment as an example of protection of a bioactive protein

Introduction:

The role of bovine colostrum as a protectant of bioactive protein activity in a gastric environment was investigated using the urease enzyme. To investigate the protective properties of colostrum, the effect of simulated gastric fluid on urease activity was compared in the presence and absence of bovine colostrum.

Materials and Methods:

Reagents

Urease: Sigma Jack Bean Urease Type III (Cat No U-1500) was used as the source of the enzyme. The freeze-dried urease (quoted activity of 16 units per mg; one unit liberates 1.0 μmole of ammonia from urea per min at pH 7.0 and 25° C.) was dissolved in MilliQ water to 0.3 units/μl (approx. 21 mg/ml) and stored on ice until used in the study.

Simulated Gastric Fluid (SGF: (adapted from Hilger et al, 2001). A 0.32% solution of Sigma porcine pepsin (Cat No P-7012 from stomach mucosa) was prepared in 0.03M NaCl and adjusted to pH 1.2 with HCl.

Colostrum: Defatted, freeze dried Bovine Colostrum Extract (Anadis Limited “Gastran” Batch G01) was sourced from non-immunised cows and a stock preparation was reconstituted at 300 mg/ml in MilliQ water. See Example 2 for method of production of Bovine Colostrum Extract.

Treatment of Urease with Simulated Gastric Fluid (SGF)

Incubation mixtures were prepared in quadruplicate by combining aliquots of the urease solution with an equal volume of either the reconstituted colostrum or MilliQ water. A control mixture of the colostrum preparation and water was also prepared (in duplicate).
Incubation mixtures 1-4: 100 μl of urease+100 μl of MilliQ water
Incubation mixtures 5-8: 100 μl of urease+100 μl of colostrum
Incubation mixtures 9-10: 100 μl of colostrum+100 μl of MilliQ water

Mixtures were preheated in a 37° C. water bath for 5 minutes before starting treatment by the addition of 50 μl of SGF to tubes 1, 2, 5, 6 and 9. Mock treatments were initiated by the addition of 50 μl of 0.03M NaCl to tubes 3, 4, 7, 8 and 10. All samples were incubated for 15 minutes at 37° C. before reactions were stopped by the addition of 0.25 volumes of chilled 160 mM Na₂CO₃ to each tube. Tubes were then centrifuged for 3 minutes at 20,800 g in an Eppendorf centrifuge 5417C and stored on ice. Supernatants were assayed for urease activity.

Urease Assay

Urease activity was assayed using a coupled enzyme assay for increased sensitivity (modified from Kaltwasser and Schlegel, 1966). In this reaction, the urease enzyme catalyses the hydrolysis of urea:
Urea+H₂O+2H⁺2NH⁴⁺+CO₂
which is measured by coupling ammonia production to a glutamic dehydrogenase reaction:
2NH⁴⁺+2 α-ketoglutarate+2 NADH [symbol] 2 glutamate+2NAD⁺+2H₂O

The reaction is followed by the oxidation of NADH to NAD.

The final assay volume of 1 ml contained final concentrations of 1.6 mM α-ketoglutarate (Boeringer Mannheim Cat No.127 205),1.5 mM NADH (Sigma β-NADH Cat No. N-8129), 15 units/ml of L-glutamic dehydrogenase (Sigma Cat No. G-4387), 10 mM Urea (Boehringer Mannheim Cat No. 100 164) and 1 mM sodium sulphide (Sigma Cat No S4766) in 50 mM Tris-HCl buffer (pH 8.0). These reagents were mixed in 1cm path length polystyrene cuvettes (Sarstedt Cat No 67.742) and then allowed to equilibrate for several minutes to room temperature in a Beckman DU70 recording spectrophotometer. The spectrophotometer was zeroed using the above mixture, before the addition of the sample.

A ten-microlitre sample of the supernatant of each incubation mixture was added to the assay mixture to start the assay. The reaction rate was recorded every 10 seconds at 340 nm for up to 4 min at RT. Reaction rate was calculated from the linear portion of curve (generally after first 1-2 mins) and urease activity was then expressed as μmole of urea hydrolysed per min per mg of protein.

Results

| | | | Urease Activity | |
|---|---|---|---|---|
| | | | (umoles urea/min/mg enzyme) | |
| Mix | Sample | Treatment | Average | SD |
| 1 - 2 | Urease + H2O | SGF | 0.027 | 0.022 |
| 3 - 4 | Urease + H2O | Mock | 0.158 | 0.021 |
| 5 - 6 | Urease + Colostrum | SGF | 0.138 | 0.018 |
| 7 - 8 | Urease + Colostrum | Mock | 0.173 | 0.014 |

| | | | Background Urease Activity |
|---|---|---|---|
| | | | (ABS 340 nm/min) |
| 9 | Colostrum + H2O | SGF | 0.021 |
| 10 | Colostrum + H2O | Mock | 0.019 |

As shown in the table of results and FIG. 1, the addition of simulated gastric fluid to a diluted urease solution resulted in the irreversible denaturation of the enzyme with subsequent loss of activity. The remaining low level of urease activity after SGF was equivalent to that found in the colostrum alone (no urease) controls. NOTE: “MOCK” TREATMENT is a treatment where saline rather than simulated gastric fluid (SGF) is used.

In contrast, the addition of colostrum to the urease solution provided protection of the urease from the effect of the pepsin and acid. In the presence of colostrum, the protected urease retained approximately 80% of the activity of the mock treated urease/colostrum mixture. Using this enzyme model, bovine colostrum has been shown to provide protection of protein activity in a simulated gastric environment.

### REFERENCES

Hilger C, Grigioni F, De Beaufort C, Michel G, Freilinger J and Hentges F (2001). Differential binding of IgG and IgA antibodies to antigenic determinants of bovine serum albumin. Clin Exp Immunol, 123:387-394.

Kaltwasser H and Schlegel H G (1966). NADH-dependent coupled enzyme assay for urease and other ammonia-producing systems. Analytical Biochem, 16:132-138.

Scott D R, Weeks D, Hong C, Postius S, Melchers K and Sachs S (1998). The role of internal urease in acid resistance of Helicobacter pylori. Gastroenterology, 114: 58-70.

### Example 2

Method of Preparation of Processed Bovine Colostrum Powder

The following diagram shows the principles used to take colostrum and convert it to a processed form.

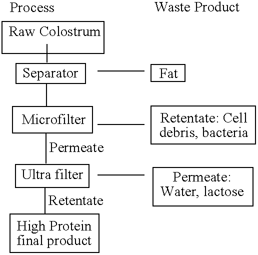

The raw colostrum is collected from dairy cows most preferably at the first milking after calving. The colostrum is stored at 4° C. on farm and then transported either for longer term storage at −20° C. or sent directly to wet manufacturing.

The raw colostrum is warmed to approximately 37° C. and then skimmed with a rotary milk separator to remove fat. The resultant liquid may be pasteurised or microfiltered with a 7-10 micron ceramic filter system (to remove bacteria and debris. The liquid is then Ultrafiltered (for example in a Abcor 10 m² Ultrafiltration plant) to remove a majority of the water, lactose and electrolytes leaving a high protein concentrate. The resultant high protein concentrate is further processed preferably by lyophilization (freeze-drying) or spray-drying.

The above method yield a processed bovine colostrum powder with the specifications as below. This product as defined below is listed as a substance suitable for inclusion in therapeutic goods by the Therapeutic Goods Authority of Australia.

Appearance: Free-flowing, pale yellow powder.

Properties: Dispersible in water. Mild odour of milk when contacted with moisture.

Moisture Range 2 to 5% m/m AS 2300.1.1 (1988)

Fat Range 1 to 4% m/m AS 2300.1.3 (1988)

Ash (@550° C.) Not more than 8% m/m AS 2300.1.5 (1988)

Total Nitrogen (TN) For information** AS 2300.1.2 (1991)
**Used to calculate the value for true protein

Non-protein nitrogen(NPN) For information** AS 2300.1.2.2 (1988)
**Used to calculate the value for true protein

True protein Not less than 60% m/m (TN-NPN) %×6.38

Protein Not less than 60% m/m AS 2300.1.2 (1991)

Lactose (monohydrate) Not more than 15% m/m
UV assay following enzymatic hydrolysis and oxidation
(Boehringer Mannheim)

Total immunoglobulins Not less than 20% m/m
Radial immunodiffusion assay
(m/m means mass of component by total mass of composition)

Microbial limits Complies with TGA guidelines

Residues: Heavy metals Agricultural and Veterinary chemicals

Subject to ANZFA Food Standards Code for dairy products. Where there is no applicable Food Standard, the BP test for heavy metals applies (2 ppm calculated as lead) and also the BP requirements for pesticide residues.

‘AS’ refers to document of the Australian Standards Organisation series of ‘Australian Standards’—in this case referring to standardised methods of quality and component testing for dairy products.

### Example 3

Colostrum Protection of Antibody Activity During Acid/Pepsin Treatment

The role of bovine colostrum as a protectant of protein activity in a gastric environment was investigated using an antibody model. To investigate the protective properties of colostrum, the effect of simulated gastric fluid on the bioactivity of an influenza virus specific monoclonal antibody was compared in the presence and absence of bovine colostrum.

Materials and Methods:

Reagents

Influenza Virus and Specific Monoclonal Antibody: The type A influenza virus used in this study was Mem71_{H}-Bel_{N} (H3N3). The antibody used was a Mem71_{H}-Bel_{N} specific IgG2a monoclonal (Mab 36/2) diluted 1:10 in phosphate buffered saline.

Simulated Gastric Fluid (SGF): (adapted from Hilger et al, 2001). A 0.32% solution of Sigma porcine pepsin (Cat No P-7012 from stomach mucosa) was prepared in 0.03M NaCl and adjusted to pH 1.2 with HCl.

Colostrum: Defatted, freeze dried Bovine Colostrum Extract (Anadis Limited “Gastran” Batch G01) was sourced from non-immunised cows and a stock preparation was reconstituted at 300 mg/mL in MilliQ water.

Treatment of Monoclonal Antibody 36/2 with SGF

Incubation mixtures were prepared by combining aliquots of antibody diluted 1:10 in phosphate buffered saline (PBS) with an equal volume of either the reconstituted colostrum or MilliQ water. A control mixture of the colostrum preparation and water was also prepared.
Incubation mixtures 1-2: 100 μl of Mab 36/2+100 μl of MilliQ water
Incubation mixtures 3-4: 100 μl of Mab 36/2+100 μl of colostrum
Incubation mixtures 5-6: 100 μl of colostrum+100 μl of MilliQ water

Mixtures were preheated in a 37° C. water bath for 5 minutes before starting treatment by the addition of 50 μl of SGF to tubes 1, 3 and 5. Mock treatments were initiated by the addition of 50 μl of 0.03M NaCl to tubes 2,4 and 6. All samples were incubated for 15 minutes at 37° C. before reactions were stopped by the addition of 0.25 volumes of chilled 160 mM Na₂CO₃ to each tube. Tubes were then centrifuged for 3 minutes at 16,100 g in an Eppendorf centrifuge 5415D and stored on ice. Supernatants were assayed for antibody activity.

Haemagglutination Inhibition Assay

Antibody activity was assayed using the haemagglutination inhibition (HI) assay. Haemagglutination titrations and HI assays were performed by standard procedures with 96 well U-bottomed microtitre plates (Sarstedt Group, SA, Australia) and 1% (vol/vol) chicken erythrocytes. The haemagglutination assay was used to quantitate the haemagglutinating units (HAU) of the virus used for the HI assay. Each incubation mixture was diluted 1:3.2 in PBS which would result in a starting concentration of 1:100 of Mab 36/2. Incubation mixtures were then serially diluted two-fold in duplicate wells across the microtitre plate for a final volume of 25 μL per well. An equal volume of virus at a concentration of 4 HAU was added to each well and antibody and virus incubated for 30 minutes at room temperature. After 30 minutes 25 μL of chicken erythrocytes (1% vol/vol in PBS) was added to each well and incubated for 30 minutes. The HI titre was taken as the reciprocal of the highest antibody titre inhibiting 4 HAU of virus. The assay was repeated twice in duplicate.

Results

| | | HI Titre | |
|---|---|---|---|
| Sample | Treatment | Average | SD |
| Mab 36/2 + H2O | SGF | 200 | 0 |
| Mab 36/2 + H20 | Mock | 1200 | 462 |
| Mab 36/2 + Colostrum | SGF | 9600 | 3695 |
| Mab 36/2 + Colostrum | Mock | 8000 | 3200 |
| Mab 36/2 (1/100 dilution) | Untreated | 800 | 0 |
| | | Background Colostrum | |
| | | Activity | |
| Colostrum + H2O | SGF | 250 | 100 |
| Colostrum + H2O | Mock | 350 | 300 |

As shown in the table of results and FIG. 2, the treatment of Mab 36/2 with simulated gastric fluid resulted in a 6-fold reduction in the haemagglutination inhibition activity of the antibody.

In contrast, the addition of colostrum to the antibody preparation provided protection of the antibody from the effect of the pepsin and acid. In the presence of colostrum, the protected antibody showed no reduction of activity. Using this antibody model, bovine colostrum has been shown to provide protection of antibody activity in a simulated gastric environment.

### REFERENCES

Hilger C, Grigioni F, De Beaufort C, Michel G, Freilinger J and Hentges F (2001). Differential binding of IgG and IgA antibodies to antigenic determinants of bovine serum albumin. Clin Exp Immunol, 123:387-394.

Deliyannis G, Jackson D, Dyer w, Bates J, Coulter A, Harling-McNabb L, Brown L, (1998). Immunopotentiation of humoral and cellular responses to inactivated influenza vaccines by two different adjuvants with potential for human use. Vaccine, 16:2058-2068.

Anders E M, Hartley C, Jackson D (1990). Bovine and mouse serum β inhibitors of influenza A viruses are mannose-binding lectins. Proc Natl Acad USA, 87:44854489.

### Example 4

Colostrum Protection of Lactobacillus plantarum Viability During Acid/Pepsin Treatment

Introduction:

The role of bovine colostrum as a protectant of bacterial viability in a gastric environment was investigated using a Lactobacillus viable plate count assay. To investigate the protective properties of colostrum, the effect of simulated gastric fluid on Lactobacillus plantarum was compared in the presence and absence of bovine colostrum.

Materials and Methods:

Reagents

Lactobacillus plantarum: The Lactobacillus plantarum strain used in this study is a strain used in probiotic and was taken from the culture collection of the Microbial Research Unit Royal Childrens Hospital, Parkville, Victoria and typed by 16S sequencing (sequencing of DNA from ribosomal subunits. The strain was cultured on horse blood agar (HBA) plates in a 37° C. C0₂ incubator for 48 to 72 hours. An inoculum of Lactobacillus was prepared by picking at least 2 colonies from a HBA plate and inoculating 2 mL of saline to reach a turbidity equivalent to a 0.5 McFarland standard. This inoculum was then used for all treatments.

Simulated Gastric Fluid (SGF): (adapted from Hilger et al, 2001). A 0.32% solution of Sigma porcine pepsin (Cat No P-7012 from stomach mucosa) was prepared in 0.03M NaCl and adjusted to pH 1.2 with HCl.

Colostrum: Defatted, freeze dried Bovine Colostrum Extract (Anadis Limited “Gastran” Batch G01) was sourced from non-immunised cows and irradiated. A stock preparation was reconstituted at 300 mg/mL in MilliQ water.

Treatment of Lactobacillus with SGF

Incubation mixtures were prepared by combining aliquots of the L. plantarum inoculum with an equal volume of either the reconstituted colostrum or MilliQ water. A control mixture of the colostrum preparation and water was also prepared.
Incubation mixtures 1-2: 100 μl of Lactobacillus+100 μl of MilliQ water
Incubation mixtures 3-4: 100 μl of Lactobacillus+100 μl of colostrum
Incubation mixtures 5-6: 100 μl of colostrum+100 μl of MilliQ water

Mixtures were preheated in a 37° C. water bath for 5 minutes before starting treatment by the addition of 50 μl of SGF to tubes 1, 3 and 5. Mock treatments were initiated by the addition of 50 μl of 0.03M NaCl to tubes 2, 4 and 6. All samples were incubated for 15 minutes at 37° C. before reactions were stopped by the addition of 0.25 volumes of chilled 160 mM Na₂CO₃ to each tube.

Lactobacillus Viable Plate Counts

Lactobacillus survival after treatment was assayed using a viable plate count technique. Ten-fold dilutions of the incubation mixtures were prepared in saline immediately after treatment. 100 μL of each dilution was then spread onto duplicate plates. Plates were incubated for 48 hours and the number of colonies per plate counted. The number of colony forming units (cfu) per mL in the incubation mixture was then calculated by multiplying the number of cfu/mL of diluted suspension by the dilution factor. The assay was done two times in duplicate.

Results

| | | Plate Count | |
|---|---|---|---|
| | | (log₁₀ cfu/mL) | |
| Sample | Treatment | Average | SD |
| Lactobacillus + H2O | SGF | 3.99 | 0.36 |
| Lactobacillus + H2O | Mock | 6.99 | 0.24 |
| Lactobacillus + Colostrum | SGF | 7.01 | 0.19 |
| Lactobacillus + Colostrum | Mock | 6.96 | 0.27 |
| | | Background Colostrum | |
| | | Activity | |
| Colostrum + H2O | SGF | 0 | 0 |
| Colostrum + H2O | Mock | 0 | 0 |

As shown in the table of results and FIG. 3, the addition of simulated gastric fluid to an inoculum of Lactobacillus resulted in a 1000 fold reduction in Lactobacillus viability.

In contrast, the addition of colostrum to the Lactobacillus inoculum provided protection of the bacteria from the effect of the pepsin and acid. In the presence of colostrum, the protected Lactobacilli showed no reduction in viability. Using this viable count technique, bovine colostrum has been shown to provide protection of probiotic viability in a simulated gastric environment.

### REFERENCES

Hilger C, Grigioni F, De Beaufort C, Michel G, Freilinger J and Hentges F (2001). Differential binding of IgG and IgA antibodies to antigenic determinants of bovine serum albumin. Clin Exp Immunol, 123:387-394.

Miles A, Misra S (1938). The estimation of the bactericidal power of the blood. J Hyg, 38:732-749.

### Example 6

Colostrum Protection of Erythomycin Activity During Acid/Pepsin Treatment

Introduction:

The role of bovine colostrum as a protectant of activity of a bioactive moiety in a gastric environment was investigated using an antibiotic model. To investigate the protective properties of colostrum, the effect of simulated gastric fluid on erythromycin activity was compared in the presence and absence of bovine colostrum.

Materials and Methods:

Reagents

Erythromycin: Boehringer Mannheim Erythromycin (C₃₇H₅₇NO₁₃) was used as the source of the antibiotic, dissolved in 95% ethanol at a stock concentration of 1 mg/mL and stored at 4° C. For this study the erythromycin stock was diluted in MilliQ water to 100 μg/mL and stored on ice until use.

Simulated Gastric Fluid (SGF): (adapted from Hilger et al, 2001). A 0.32% solution of Sigma porcine pepsin (Cat No P-7012 from stomach mucosa) was prepared in 0.03M NaCl and adjusted to pH 1.2 with HCl.

Colostrum: Defatted, freeze dried Bovine Colostrum Extract (Anadis Limited “Gastran” Batch G01) was sourced from non-immunised cows and a stock preparation was reconstituted at 300 mg/mL in MilliQ water.

Treatment of Erythromycin with SGF

Incubation mixtures were prepared by combining aliquots of the erythromycin solution with an equal volume of either the reconstituted colostrum or MilliQ water. A control mixture of the colostrum preparation and water was also prepared.
Incubation mixtures 1-2: 100 μl of erythromycin+100 μl of MilliQ water
Incubation mixtures 3-4: 100 μl of erythromycin+100 μl of colostrum
Incubation mixtures 5-6: 100 μl of colostrum+100 μl of MilliQ water

Mixtures were preheated in a 37° C. water bath for 5 minutes before starting treatment by the addition of 50 μl of SGF to tubes 1, 3 and 5. Mock treatments were initiated by the addition of 50 μl of 0.03M NaCl to tubes 2,4 and 6. All samples were incubated for 15 minutes at 37° C. before reactions were stopped by the addition of 0.25 volumes of chilled 160 mM Na₂CO₃ to each tube. Tubes were then centrifuged for 3 minutes at 16,100 g in an Eppendorf centrifuge 5415D and stored on ice. Supernatants were assayed for erythromycin activity.

Erythromycin Susceptibility Assay

Erythromycin activity was assayed using a Bacillus subtilis disc diffusion susceptibility test. An inoculum of B. subtilis (ATCC 6633) was prepared by picking at least 2 colonies from an overnight culture grown on horse blood agar (HBA) and inoculating 2 mL of saline to reach a turbidity equivalent to a 0.5 McFarland standard. HBA plates for the assay were then inoculated by streaking a sterile swab, dipped into the standardised solution, evenly in three directions over the entire surface of the plate to obtain a uniform inoculum. Plates were then allowed to dry for 3 to 5 minutes before the discs were applied.

Each treatment was serially diluted 1:2 with MillQ water, resulting in six dilutions for each. 20 μL of each dilution was then loaded onto duplicate blank susceptibility discs (Oxoid, Hampshire, England). These discs were allowed to dry for at least 30 minutes before being placed onto duplicate plates. Each plate contained six evenly placed discs corresponding to the six dilutions of a single treatment. Untreated erythromycin diluted in MilliQ water to the equivalent concentration of the treated samples was also used as a control to obtain a standard curve. Plates were incubated for 16-18 hours at 37° C.

After 16-18 hours incubation the susceptibility of B. subtilisto erythromycin was determined by measuring the diameter of the zones of inhibition which appear around the discs. These zones result from the diffusion of the antibiotic from the disc into the surrounding agar. A standard curve was generated using the diameters of zones resulting from the serially diluted untreated erythromycin control. Diameters for the test samples were then used to obtain the percentage of erythromycin activity remaining compared with the untreated control. The assay was repeated three times in duplicate.

Results

| | | Erythromycin Activity | |
|---|---|---|---|
| | | (% compared with untreated control) | |
| Sample | Treatment | Average | SD |
| Erythromycin + | SGF | 8 | 3 |
| H2O | | | |
| Erythromycin + | Mock | 114 | 14 |
| H20 | | | |
| Erythromycin + | SGF | 140 | 20 |
| Colostrum | | | |
| Erythromycin + | Mock | 123 | 9 |
| Colostrum | | | |
| Erythromycin + | Untreated | 100 | 0 |
| H20 | | | |

| | | Background Erythromycin |
|---|---|---|
| | | Activity |
| | | (%) |
| Colostrum + H2O | SGF | 0 |
| Colostrum + H20 | Mock | 0 |

As shown in the table of results and FIG. 4, the addition of simulated gastric fluid to a 100 μg/mL solution resulted in a 92% reduction in erythromycin activity.

In contrast, the addition of colostrum to the erythromycin solution provided protection of the erythromycin from the effect of the pepsin and acid. In the presence of colostrum, the protected erythromycin retained 100% activity and actually showed improved activity when compared with the untreated control (see FIGS. 1 and 2). This enhanced activity was also evident for the solutions of erythromycin and colostrum that were mock treated. Colostrum alone showed no background antibiotic activity so the enhanced activity seen with added colostrum cannot be explained by background erythromycin present in the colostrum. Using this antibiotic model, bovine colostrum has been shown to provide protection of antibiotic activity in a simulated gastric environment.

### REFERENCES

Hilger C, Grigioni F, De Beaufort C, Michel G, Freilinger J and Hentges F (2001). Differential binding of IgG and IgA antibodies to antigenic determinants of bovine serum albumin. Clin Exp Immunol, 123:387-394.

Barry A L and Thornsberry C (1991). Susceptibility tests: diffusion test procedures. In Balos A, Hauser W J, Herman K L, Isenberg H D, and Shadomy H J. Manual of Clinical Microbiology 5^{th} Edition, American Society for Microbiology, Washington, pp 1117-1125.

The British Society for Antimicrobial Chemotherapy (1991) A Guide to Sensitivity Testing. The British Society for Antimicrobial Chemotherapy, San Diego.

### Example 7

Recolostrum Protection of Lactobacillus casei shirota Viability During Acid/Pepsin Treatment

Introduction:

The role of bovine colostrum as a protectant of probiotic viability in a gastric environment was investigated using a Lactobacillus viable plate count assay. To investigate the protective properties of colostrum, the effect of simulated gastric fluid on Lactobacillus casei shirota isolated from Yakult fermented liquor was compared in the presence and absence of bovine colostrum.

Materials and Methods:

Reagents

Lactobacillus casei shirota: The Lactobacillus casei shirota strain used in this study was isolated from the probiotic product—Yakult. The strain was cultured on horse blood agar (HBA) plates in a 37° C. CO₂ incubator for 48 to 72 hours. An inoculum of Lactobacillus was prepared by picking at least 2 colonies from a HBA plate and inoculating 2 mL of saline to reach a turbidity equivalent to a 0.5 McFarland standard. This inoculum was then used for all treatments.

Simulated Gastric Fluid (SGF): (adapted from Hilger et al, 2001). A 0.32% solution of Sigma porcine pepsin (Cat No P-7012 from stomach mucosa) was prepared in 0.03M NaCl and adjusted to pH 1.2 with HCl.

Colostrum: Defatted, freeze dried Bovine Colostrum Extract (Anadis Limited “Gastran” Batch G01) was sourced from non-immunised cows and irradiated. A stock preparation was reconstituted at 300 mg/mL in MilliQ water.

Treatment of Lactobacillus with SGF

Incubation mixtures were prepared by combining aliquots of the L. casei shirota inoculum with an equal volume of either the reconstituted colostrum or MilliQ water. A control mixture of the colostrum preparation and water was also prepared.
Incubation mixtures 1-2: 100 μl of Lactobacillus+100 μL of MilliQ water
Incubation mixtures 3-4: 100 μl of Lactobacillus+100 μL of colostrum
Incubation mixtures 5-6: 100 μl of colostrum+100 μL of MilliQ water

Mixtures were preheated in a 37° C. water bath for 5 minutes before starting treatment by the addition of 50 μL of SGF to tubes 1, 3 and 5. Mock treatments were initiated by the addition of 50 μL of 0.03M NaCl to tubes 2, 4 and 6. All samples were incubated for 15 minutes at 37° C. before reactions were stopped by the addition of 0.25 volumes of chilled 160 mM Na₂CO₃ to each tube.

Lactobacillus Viable Plate Counts

Lactobacillus survival after treatment was assayed using a viable plate count technique. Ten-fold dilutions of the incubation mixtures were prepared in saline immediately after treatment. 100 μL of each dilution was then spread onto duplicate plates. Plates were incubated for 48 hours and the number of colonies per plate counted. The number of colony forming units (cfu) per mL in the incubation mixture was then calculated by multiplying the number of cfu/mL of diluted suspension by the dilution factor. The assay was done two times in duplicate.

Results

| | | Plate Count | |
|---|---|---|---|
| | | (log₁₀ cfu/mL) | |
| Sample | Treatment | Average | SD |
| Lactobacillus + H2O | SGF | 1.46 | 2.06 |
| Lactobacillus + H2O | Mock | 7.43 | 0.09 |
| Lactobacillus + Colostrum | SGF | 7.01 | 0.18 |
| Lactobacillus + Colostrum | Mock | 6.97 | 0.03 |
| | | Background Colostrum | |
| | | Activity | |
| Colostrum + H2O | SGF | 0 | 0 |
| Colostrum + H2O | Mock | 0 | 0 |

As shown in the table of results and FIG. 5, the addition of simulated gastric fluid to an inoculum of Lactobacillus casei shirota resulted in at least a 10,000 fold reduction in Lactobacillus viability.

In contrast, the addition of colostrum to the Lactobacillus inoculum provided protection of the probiotic bacteria from the effect of the pepsin and acid. In the presence of colostrum, the protected Lactobacilli showed no reduction in viability. Using this viable count technique, bovine colostrum has been shown to provide protection of probiotic viability in a simulated gastric environment.

### REFERENCES

Hilger C, Grigioni F, De Beaufort C, Michel G, Freilinger J and Hentges F (2001). Differential binding of IgG and IgA antibodies to antigenic determinants of bovine serum albumin. Clin Exp Immunol, 123:387-394.

Miles A, Misra S (1938). The estimation of the bactericidal power of the blood. J Hyg, 38:732-749.

### Example 8

Colostrum Protection of Cholera Toxin Activity During Acid/Pepsin Treatment

Introduction:

The role of bovine colostrum as a protectant of adjuvant activity in a gastric environment was investigated using a mucosal adjuvant. To investigate the protective properties of colostrum, the effect of simulated gastric fluid on the activity of cholera toxin was compared in the presence and absence of bovine colostrum.

Materials and Methods:

Reagents

Cholera toxin and Y1 adrenal cells: Sigma Vibrio cholerae Cholera Toxin (Cat No C-8052) was used as the source of the adjuvant. The cholera toxin was diluted in MilliQ water to a concentration of 3.12 μg/mL and stored on ice until use. Y1 mouse adrenal cells were seeded in 96-well microtitre plates at a concentraion of 2×10⁴ cells per well in DMEM growth medium supplemented with 10% foetal calf serum (FCS) and gentamicin.

Simulated Gastric Fluid (SGF): (adapted from Hilger et al, 2001). A 0.32% solution of Sigma porcine pepsin (Cat No P-7012 from stomach mucosa) was prepared in 0.03M NaCl and adjusted to pH 1.2 with HCl.

Colostrum: Defatted, freeze dried Bovine Colostrum Extract (Anadis Limited “Gastran” Batch G01) was sourced from non-immunised cows and a stock preparation was reconstituted at 300 mg/mL in MilliQ water.

Treatment of Cholera Toxin with SGF

Incubation mixtures were prepared by combining aliquots of the 3.12 μg/mL cholera toxin stock with an equal voulme of either the reconstituted colostrum or MilliQ water. A control mixture of the colostrum preparation and water was also prepared.
Incubation mixtures 1-2: 100 μL of cholera toxin+100 μL of MilliQ water
Incubation mixtures 3-4: 100 μL of cholera toxin+100 μL of colostrum
Incubation mixtures 5-6: 100 μL of colostrum+100 μL of MilliQ water

Mixtures were preheated in a 37° C. water bath for 5 minutes before starting treatment by the addition of 50 μL of SGF to tubes 1, 3 and 5. Mock treatments were initiated by the addition of 50 μL of 0.03M NaCl to tubes 2, 4 and 6. All samples were incubated for 15 minutes at 37° C. before reactions were stopped by the addition of 0.25 volumes of chilled 160 mM Na₂CO₃ to each tube. Tubes were then centrifuged for 3 minutes at 16,100 g in an Eppendorf centrifuge 5415D and stored on ice. Supernatants were filter sterilised and then assayed for toxin activity.

Y1 Adrenal Cell Bioassay

Cholera toxin activity was assayed using the Y1 adrenal cell bioassay. Y1 mouse adrenal cells were seeded in 96-well microtitre plates at a concentration of 2×10⁴ cells per well. After 48 hours, cells were washed 3 times with PBS. The growth medium was replaced with DMEM supplemented with 1% FCS and gentamicin (100 μL), containing duplicate samples of the treatment tubes, serially diluted 2-fold after an initial dilution of 1:10. Cholera toxin causes Y1 adrenal cells to change from their usual elongated morphology to a more rounded shape. These changes are concentration dependent. The cells were incubated for 18 hours and then examined for typical rounding by using a phase contrast microscope. The end point of the assay was defined as the highest dilution that showed more than 50% cell rounding. The assay was repeated twice in duplicate. Two-fold dilutions of cholera toxin at a starting concentration of 10 ng/mL were assayed in the same plates and the results used to determine the sensitivity of the assay.

Results

| | | Toxicity Titre | |
|---|---|---|---|
| | | (log 2) | |
| Sample | Treatment | Average | SD |
| Cholera toxin + H2O | SGF | 0 | 0 |
| Cholera toxin + H2O | Mock | 8 | 0 |
| Cholera toxin + | SGF | 8 | 0 |
| Colostrum | | | |
| Cholera toxin + | Mock | 7.5 | 0.71 |
| Colostrum | | | |
| Cholera toxin (10 ng/mL) | Untreated | 8 | 0 |
| | | Background Colostrum | |
| | | Activity | |
| Colostrum + H2O | SGF | 2.5 | 0.71 |
| Colostrum + H2O | Mock | 3 | 0 |

As shown in the table of results and FIG. 6, the treatment of cholera toxin with simulated gastric fluid resulted in the complete abrogation of the biological activity of cholera toxin.

In contrast, the addition of colostrum to the cholera toxin preparation provided protection of the toxin from the effect of the pepsin and acid. In the presence of colostrum, the protected cholera toxin showed no reduction of activity. Using this cholera toxin assay, bovine colostrum has been shown to provide protection of mucosal adjuvant in a simulated gastric environment.

### REFERENCES

Hilger C, Grigioni F, De Beaufort C, Michel G, Freilinger J and Hentges F (2001). Differential binding of IgG and IgA antibodies to antigenic determinants of bovine serum albumin. Clin Exp Immunol, 123:387-394.

Tauschek M, Gorrell R, Strugnell R, Robins-Browne R (2002). Identification of a protein secretory pathway for the secretion of het-labile enterotoxin by an enterotoxigenic strain of Escherichia coli. Proc Natl Acad Sci USA, 99:7066-7071.

### Example 9

Protection of Lactobacillus plantarum in an In Vivo Mouse Model

Introduction:

The role of bovine colostrum as a protectant of probiotic viability in a gastric environment was investigated using an in vivo mouse model. To investigate the protective properties of colostrum mice were fed Lactobacillus plantarum with without colostrum, or as a positive control, sodium bicarbonate.

Materials and Methods:

Reagents

Lactobacillus plantarum: The Lactobacillus plantarum strain used in this study was the strain referred to in Example 4. The strain was cultured on horse blood agar (HBA) plates in a 37° C. CO₂ incubator for 48 to 72 hours. An inoculum of Lactobacillus was prepared by harvesting a confluent lawn of bacteria from at least 2 plates and inoculating 50 mL of saline to reach a turbidity equivalent to a McFarland 4 standard. The bacteria were pelleted and the supernatant discarded. The bacterial pellet was then resuspended in 2.5 mL of saline. This inoculum was mixed 1:1 with bioshield, sodium bicarbonate or saline to give a final bacterial concentration of 1×10⁹ cfu/mL.

Colostrum Extract (also designated as bioshield): Defatted, freeze dried Bovine Colostrum Extract (Anadis Limited “Gastran” Batch G01) was sourced from non-immunised cows and irradiated. Sample preparations were reconstituted at 20, 40, and 100 mg/mL in MilliQ water.

Sodium bicarbonate: Sodium bicarbonate was also reconstituted at concentrations of 20, 40 and 100 mg/mL in MilliQ water.

Infection of Mice with Lactobacillus

Mice were deprived of food for 3 hours prior to inoculation to ensure that their stomachs were empty. The mice were then inoculated by gavage with 100 μL of each preparation, an inoculum of ˜1×10⁸ cfu of Lactobacillus (the actual numbers of Lactobacilli in each preparation was determined by viable plate counts). There were 7 groups of mice, each with 3 mice per group, the mice were given the following preparations:
Lactobacillus+saline
Lactobacillus+20 mg/mL bioshield
Lactobacillus+40 mg/mL bioshield
Lactobacillus+100 mg/mL bioshield
Lactobacillus+20 mg/mL sodium bicarbonate
Lactobacillus+40 mg/mL sodium bicarbonate
Lactobacillus+100 mg/mL sodium bicarbonate

Mice were deprived of food for the course of the experiment but were allowed autoclaved water ad libitum. Protection of Lactobacillus in vivo was assayed by counting the number of L. plantarum colonising the large intestine after 3 hours. The large intestine (caecum and colon) was harvested aseptically from each mouse into 2 mL of sterile saline. Samples were weighed and homogenised and ten-fold dilutions prepared in saline. 100 μL of each dilution was spread onto a Lactobacillus selective media (horse blood agar containing 10 μg/mL vancomycin and 12.5 μg/mL polymyxin B). Plates were incubated for 48 hours in 5% CO₂ and the number of colonies per plate counted. Lactobacillus plantarum were readily distinguishable from other Lactobacilli normally present in the normal flora of the mouse due to their opaque white appearance. The number of colony forming units (cfu) per gram of intestine was calculated.

Results

As shown in FIG. 7, survival of Lactobacillus plantarum increased in the presence of both bioshield and sodium bicarbonate. Higher concentrations of either bioshield or sodium bicarbonate resulted in increased survival and therefore better protection.

### Example 10

Colostrum Protection of Lactobacillus plantarum Viability Under Humid Conditions

Introduction:

The role of an extract of bovine colostrum, as a protectant of probiotic viability in different humidity conditions, was investigated using a Lactobacillus viable plate count assay. To investigate the protective properties of colostrum extract, the viability of Lactobacillus plantarum was compared after storage for 14 days in three constant humidity environments.

Materials and Methods:

Reagents

Lactobacillus plantarum: The Lactobacillus plantarum (L.p.) strain used in this study was the strain referred to in Example 4. The strain was cultured on horse blood agar (HBA) plates in a 37° C. CO₂ incubator for 48 to 72 hours. An inoculum of L.p. was prepared by picking at least 2 colonies from a HBA plate and inoculating 2 ml of saline to reach a turbidity equivalent to a 0.5 McFarland standard. This inoculum was then used for all treatments.

Freeze Drying Medium: The freeze-drying medium (FDM) used in these experiments was derived from Conrad et al (2000). A 2×-concentrated FDM was prepared using 40% w/v α,α-trehalose dihydrate (Sigma) and 5.7% w/v sodium tetraborate decahydrate (Sigma) in sterile 0.6 mM potassium phosphate pH 7.2. The pH of this FDM was initially adjusted to pH 6.5 with solid citric acid (Sigma) and then changed to pH 8.5 with ammonium hydroxide (Sigma 29.5%). The 2× concentrated FDM was then sterile filtered to 0.2 um.

Colostrum: Defatted, freeze-dried Bovine Colostrum Extract (Anadis Limited “Gastran” Batch G01) was sourced from non-immunised cows and a stock preparation was reconstituted at 40 mg/ml in 2× concentrated FDM.

Preparation of samples: Four aliquots of each of the following mixtures were made prior to freeze drying:
(a) L.p.+2× FDM (0.5 ml+0.5 ml)
(b) L.p.+40 mg/ml colostrum in 2× FDM (0.5 ml+0.5 ml)

These samples were mixed and then incubated at room temperature for 1 hr and then frozen on dry ice. Samples were then freeze-dried overnight.

Stability of Lactobacillus plantarum at different relative humidities Three constant humidity chambers were set up using sealed boxes containing different solutions. A saturated solution of LiCl (Sigma) maintained the relative humidity (RH) at 11.3% (water activity a_{w}=0.113). An 80% w/w solution of glycerol (BDH Analar) in water produced a 50% RH (a_{w}=0.50) and a saturated KCl (BDH Analar) solution maintained an RH of 85% (a_{w}=0.85) (refer to Forney & Brandl 1992 and Merck Index). After freeze drying, each sample was gently ground to a fine powder using a mortar and pestle. Two samples (L.p.+FDM and L.p.+Colostrum in FDM) were incubated in open wide-mouth vials in each chamber, for 14 days at 20° C. A control sample of each mixture was stored at 20° C. in closed vials.

Lactobacillus Viable Plate Counts

Lactobacillus survival, after incubation at different relative humidities, was assayed using a viable plate count technique. Ten-fold dilutions of the incubation mixtures were prepared in saline containing 0.05% of the surfactant Tween 20. 100 μl of each dilution was then spread onto duplicate plates. Plates were incubated for 48 hours and the number of colonies per plate counted. The number of colony forming units (cfu) per ml in the incubation mixture was then calculated by multiplying the number of cfu/ml of diluted suspension by the dilution factor. These counts were then expressed as a percentage of the control sample.

Results

| Relative | | |
|---|---|---|
| Humidity | Sample | Survival of L. p |
| 11.3% | L. p + FDM + Colostrum | 100% |
| 11.3% | L. p + FDM | 99.3% |
| 50% | L. p + FDM + Colostrum | 61.3% |
| 50% | L. p + FDM | 36.7% |
| 85% | L. p + FDM + Colostrum | 18.1% |
| 85% | L. p + FDM | 8.5% |

An extract of bovine colostrum increased the survival of a freeze-dried probiotic, Lactobacillus plantarum, during exposure to medium and high humidity conditions.

### REFERENCES

Conrad P B, Miller D P, Cielenski P R and de Pablo J J (2000). Stabilization and Preservation of Lactobacillus acidophilus in Saccharide Matrices. Cryobiology 41:17-24.

Forney C F and Brandl D G (1992). Control of Humidity in Small Controlled-environment Chambers using Glycerol-Water Solutions. HortTechnology Jan/Mar 2(1): 52-54.

Merck Index: Misc-98 “Saturated Solutions” and Misc-103 “Constant Humidity Solutions”.

Finally, it is understood that various other modifications and/or alterations may be made without departing from the spirit of the present invention as outlined herein.

1. A method of improving the viability of a labile bioactive substance on administration to a hostile environment, the method comprising forming a mixture of the bioactive substance and mammalian colostrum.

2. A method according to claim 1 wherein bioactive substance shows at least 20% diminution in function after the substance has been incubated for sixty minutes at 37° C. in a liquor comprising a 0.32% solution of porcine pepsin in 0.03 M, NaCL and adjusted to pH 1.2 with HCL.

3. A method according to claim 1 wherein the mixture is administered via the gastrointestinal or respiratory tract.

4. A method according to claim 1 wherein the mixture is administered orally.

5. A method according to claim 1 wherein the bioactive substance is selected from the group consisting of growth promoters, antineoplastic agents, oral vaccines, inhalants, living microorganisms (for example protobiotics such as Lactobacillus spp), peptides, polypeptides, nucleotides, polynucleotides, nucleosides, proteins, glycoproteins, sugars and complex carbohydrates, anti-infectants, antimicrobials, disinfectants, antiseptics, antidepressants, psychoactive agents, genetically modified organisms and infectious agents used as vectors for other bioactive substances eg bacterial vectors (including E. coli, Salmonella, Vibrio, Lactobacilli, Bacillus, Mycobacteria, Shigella), viral vectors (including Adenovirus, Poxvirus, Bacculovirus, Herpesvirus, Enterovirus, Paramyxovirus and Orthomyxovirus), plant vectors (including tobacco, potato and banana), yeast vectors, immunoglobulins, affinity purified immunoglobulins including antibodies directed against diseases and disease causing agents (for example Helicobacter pylori, E. coli, Bacillus spp, pathogenic Yersinia spp., and allergens) and fragments, derivatives and complexes containing any of the above.

6. It is particularly preferred that the bioactive material comprises one or more of immunoglobulins, monoclonal or polyclonal antibodies, chimeric antibodies, humanised monoclonal antibodies and dendrimer presented immunoactive fragments or immunoactive fragments.

7. A method according to claim 1 wherein the bioactive material comprises immunoactive fragments selected from the group consisting of F(ab) and F(ab)₂ fragments, recombinant immunoactive fragements, affinity purified immunoglubulins and immunoactive fragments thereof and mixtures.

8. A method according to claim 1 wherein the bioactive substance comprises at least one probiotic.

9. A method according to claim 8 wherein the bioactive substance comprises one or more of Lactobacillus spp and Bifidobacter spp.

10. A method according to claim 1 wherein the bioactive substance is selected from the group consisting of:
(a) antibodies and fragments thereof against the disease organisms H. pylori, enterotoxic E. coli (ETEC), picorna viruses especially Enteroviruses rotavirus anthrax and Yersinia pestis;
(b) antibodies and fragments thereof against toxins produced by H. pylori, ETEC, anthrax, Yersinia pestis and antibodies against ricin; and
(c) oral vaccines and inhalation vaccines against H. pylori, ETEC, picorna viruses especially Enteroviruses, rotavirus, anthrax, Yersinia pestis.

11. A method according to claim 10 wherein the bioactive substance is selected from oral vaccines or antibodies or antibody fragments against H. pylori or enterovirus 71.

12. A method according to claim 1 wherein the bioactive substance comprises polyclonal antibodies or fragments thereof derived from colostrum or avian eggs.

13. A method according to claim 1 wherein the bioactive material comprises acid labile antibiotics.

14. A method of treatment or prophylaxis of gastrointestinal disease in a patient comprising orally administering to the patient a composition comprising a mixture of (a) a vaccine directed against a pathogenic microorganism selected from H. pylori and coliforms and (b) mammalian colostrum.

15. A composition according to claim 1 wherein the weight ratio of colostrum to active substance is greater than 0.5:1.

16. A composition according to claim 15 wherein said weight ratio is greater than 2:1.

17. A method according to claim 1 wherein the mixture is administered in a composition comprising one or more carriers or excipients suitable for administration orally or by inhalation.

18. A method according to claim 1 wherein the colostrum comprises bovine colostrum retained from no more than two days post parturition.

19. A method according to claim 18 wherein the bovine colostrum comprises colostrum retained from the day post parturition.

20. A method according to claim 1 wherein a liquid mixture of colostrum and bioactive substance is formed and the liquid mixture is dried to form a solid.

21. A method according to claim 1 wherein the mammalian colostrum is dried.

22. A method according to claim 7 wherein the ratio of IgG to total protein in the colostrum collected is at least 10%.

23. A composition for administration of a labile bioactive substance the composition comprising a mixture of the bioactive substance and mammalian colostrum.

24. A composition according to claim 23 wherein the bioactive substance is selected from the group consisting of growth promoters, antineoplastic agents, oral vaccines, inhalants, living microorganisms (for example protobiotics such as Lactobacillus spp), peptides, polypeptides, nucleotides, polynucleotides, nucleosides, proteins, glycoproteins, sugars and complex carbohydrates, anti-infectants, antimicrobials, disinfectants, antiseptics, antidepressants, psychoactive agents, genetically modified organisms and infectious agents used as vectors for other bioactive substances eg bacterial vectors (including E. coli, Salmonella, Vibrio, Lactobacilli, Bacillus, Mycobacteria, Shigella), viral vectors (including Adenovirus, Poxvirus, Bacculovirus, Herpesvirus, Enterovirus, Paramyxovirus and Orthomyxovirus), plant vectors (including tobacco, potato and banana), yeast vectors, immunoglobulins, affinity purified immunoglobulins including antibodies directed against diseases and disease causing agents (for example Helicobacter pylori, E. coli, Bacillus spp, pathogenic Yersinia spp., and allergens) and fragments, derivatives and complexes containing any of the above.

25. A composition according to claim 23 wherein the bioactive substance is selected from the groups consisting of:
(a) antibodies and fragments thereof against the disease organsims H. pylori, enterotoxic E. coli (ETEC), picoma viruses especially Enteroviruses rotavirus anthrax and Yersinia pestis;
(b) antibodies and fragments thereof against toxins produced by H. pylori, ETEC, anthrax, Yersinia pestis and antibodies against ricin; and
(c) oral vaccines and inhalation vaccines against H. pylori, ETEC, picorna viruses especially Enteroviruses, rotavirus, anthrax, Yersinia pestis.

26. A composition according to claim 23 wherein the bioactive substance is selected from antibiotics, probiotics, immunoglubins, immunoglubilin fragments and mixtures thereof.

27. A composition according to claim 23 wherein the weight ratio of colostrum to bioactive substance is greater than 2:1.

28. A composition according to claim 23 wherein the mammalian colostrum comprises bovine colostrum retained from no more than two days post parturition.

29. A composition according to claim 23 wherein the mixture is formed by drying an intimate mixture of the bioactive substance and mammalian colostrum.

30. Use of mammalian colostrum in the manufacture of a medicament for treatment or prophylaxis of disease wherein the bovine colostrum is mixed with a labile bioactive substance for treatment of the disease whereby the activity of the bioactive substance is more fully preserved.
